(19) United States
(12) Patent Application Publication
(10) Pub. No.: US 2019/0336787 A1
(43) Pub. Date: Nov. 7, 2019

(54) MASK APPARATUS FOR SKIN BEAUTY AND MASK APPARATUS FOR SKIN BEAUTY WITH COMPLEX LAYER INCLUDING GOLD AND SILVER

(71) Applicant: JIN YOUNG R&S CO., LTD, Daegu (KR)

(72) Inventors: Hyeuk-Jeon KWEON, Daegu (KR); Sang-Mok LEE, Daegu (KR)

(21) Appl. No.: 16/226,297

(22) Filed: Dec. 19, 2018

(30) Foreign Application Priority Data
May 4, 2018 (KR) 10-2018-0051577

(51) Int. Cl. A61N 5/06; A45D 44/00; A61K 8/02; A61K 8/19

(52) U.S. Cl. CPC A61N 5/0616; A45D 44/002; A61K 8/0212; A61K 8/19; A61B 2018/00452; A61N 2005/0632; A61N 2005/0647; A61N 2005/0659; A61N 5/0625

(57) ABSTRACT

Disclosed herein is a mask apparatus for skin beauty capable of improving skin health. The mask apparatus according to the present invention comprises a face mask, a mask pad placed on the mask pad and providing skin beauty effect on a face of a user using heat and far-infrared rays, and a control unit providing currents to the mask pad and emitting heat and far-infrared rays toward the face of the user.

### CROSS REFERENCE TO RELATED APPLICATIONS PARAGRAPH

The present invention claims priority to and the benefit of Korean Patent Application No. 10-2018-0051577 filed in the Korean Intellectual Property Office on May 4, 2018, the entire contents of which are incorporated herein by reference.

### TECHNICAL FIELD

The present invention relates to a mask for skin beauty providing skin beauty effect employing far-infrared rays and heat and a mask apparatus for skin beauty with complex layer including gold and silver.

### BACKGROUND ART

Generally, skin care apparatus are classified into electrosurgical instruments, fat sucking apparatus, micro-ablation surgery apparatus, phototheraphy apparatus, cryotheraphy apparatus, treatment apparatus using laser, and the like.

These skin care apparatus employ precious metals. Typical precious metals are gold (Au) and silver (Ag).

Gold (Au) and silver (Ag) have been used in the field of health and skin beauty from ancient times.

A botanical list discloses that gold improves chronic skin disease and infections of injury as and facilitates blood circulation to be effective in skin beauty.

There was a record that gold was used in making cosmetics in Tang Dynasty. Also, it was reported that Cleopatra or Yang juei-fei used gold as skin beauty materials. The reason for this is that gold performs a function to purify, detoxicate skins and improve skin tones.

In addition, gold helps to generate collagen by activating hormone secretion and has detoxication effect by absorbing and discharging wastes, thereby improving skin tones and making skins elastic in order to improve dark complexion. Also, gold is helpful to prevent aging and resolving cellulite to be helpful in forming an oval face.

Silver has been used as sterilizers over 1,200 years. In the past, silver has prevented infections of disease using silver cups, silver dishes, and so forth and protected foods from going rancid using silver boxes. Now, silver has been used as sterilization or disinfection by putting silver coins into plates and employed in curing burns using silver powder.

NASA (National Aeronautics and Space Administration) adopts a water purifier using silver and more than half of world airline companies adopt water purifiers using silver. Accordingly, silver has been recognized as sterilizing in the world.

In addition, silver protects various kinds of disease and helps treatments to have various effects such as resistance enhancement with respect to disease, fatigue recovery, constipation control, anti-inflammation, pains, blood pressure regulation, and skin improvement.

In a recent, there is a trend for hairdressing and beauty care apparatus using light ray to be widely spread according to a great attention with respect to skin soothing, skin beauty, skin-pore care, and the like.

Hairdressing and beauty care apparatus using light ray in hospitals or skin care shops as well as at home have been manufactured and sold.

Among them, hairdressing and beauty care masks using LED are mainly used. These masks are suitable for caring skins because they is applicable depending on a skin condition according to each of wavelengths of LED.

However, a conventional face mask apparatus for skin beauty using a light has a side effect such as nystagmus caused by electromagnetic waves of LED and has an effectiveness due to light but lacks blood circulation effect and high absorption of cosmetics by heat.

Therefore, masks for skin beauty using heat and far-infrared ray have been required in order to additionally obtain the effectiveness by including gold and silver and by heat.

### SUMMARY OF THE INVENTION

It is therefore an object of the present invention to provide a face mask apparatus having thermotheraphy effect and far-infrared ray effect using heat and far-infrared rays.

It is another object of the present invention to provide a face mask for facial skin therapeutic, which is easily wearable and capable of extending his/her field of activity by securing view when wearing.

It is still a further object of the present invention to provide a face mask apparatus having effective facial skin therapeutic by cutting a loss of far-infrared ray as well as skin activation and antibacterial function by gold and silver.

It is still a further object of the present invention to provide a fabric mask for improving blood circulation and activating skin on a face skin by gold as well as providing antibacterial function on a face by silver through a mask pad including gold and silver or a fabric mask.

It is still a further object of the present invention to provide a face mask apparatus for differently providing currents depending on a skin condition of a face to provide optimal heat and far-infrared rays onto the face.

It is still a further object of the present invention to provide a face mask apparatus analyzing facial skin condition of users through only a face image to provide optimal beauty and therapeutic effect to a corresponding portion when the facial image of users are provides by interworking with an external communication device.

Pursuant to embodiments of the present invention provides a mask apparatus for skin beauty comprising a face mask, a mask pad placed within the face mask and providing skin beauty effect on a face of a user using heat and far-infrared rays, and a control unit for capable of differently providing a current to a specific part of the mask pad and differently emitting the heat and far-infrared rays onto the face of the user.

Pursuant to embodiments of the present invention, a remote control connected to the mask pad and spaced apart from the mask pad is further included. The control unit is formed in the remote control and currents are provided through the remote control to the mask pad.

Pursuant to embodiments of the present invention, a power supply formed in the remote control is further included.

Pursuant to embodiments of the present invention, a battery formed in the face mask is further included. The control unit is formed in the face mask and differently provides currents from the battery to the specific part of the mask pad to differently emit the heat and far-infrared rays onto the face of the user.

Pursuant to embodiments of the present invention, The mask apparatus further includes a fabric facial mask including gold and silver. The fabric facial mask removes skin wastes and improves a blood circulation through the heat and far-infrared rays from the mask pad by gold and directly performs a sterilizing function by silver on a facial skin.

Pursuant to embodiments of the present invention, the mask pad comprises a far-infrared ray generating pad for generating the heat and far-infrared rays and a far-infrared ray reflecting pad for reflecting the heat and far-infrared rays. The far-infrared ray generating pad is formed on one side of the face mask and the far-infrared ray reflecting pad is formed on one side of the far-infrared ray generating pad, and the far-infrared ray reflecting pad reflects the heat and far-infrared rays created from the far-infrared ray generating pad toward the face of the user.

Pursuant to embodiments of the present invention, the far-infrared ray generating pad is a planar heater and comprises a first flexible substrate layer, a heat generating layer printed on the first flexible substrate layer for silver paste to have a first pattern and generating heat when currents are provided, and a far-infrared rays generating layer printed for carbon-based graphite and Carbon Nano Tube (CNT) to have a second pattern on the heat generating layer and generating far-infrared rays using heat from the heat generating layer.

Pursuant to embodiments of the present invention, the far-infrared ray generating pad includes two or more far-infrared ray generating parts. The two or more far-infrared ray generating parts are separated each other.

Pursuant to embodiments of the present invention, reflecting pad includes a brow-region far-infrared ray generating part, an eye-region far-infrared ray generating part, and a nasolabial-line region far-infrared ray generating part, which are separated each other.

Pursuant to embodiments of the present invention, the far-infrared ray reflecting pad includes a second flexible substrate layer and a far-infrared ray reflecting layer in which silver paste is formed on the second flexible layer.

Pursuant to embodiments of the present invention, a gold deposition layer formed on the far-infrared ray reflecting layer is further included.

Pursuant to embodiments of the present invention, an input unit for inputting a control signal of the control unit is further included. The input unit generates the control signal on a manual operation or sound.

Pursuant to embodiments of the present invention, a communication unit and a remote control connected to the mask pad, including a control unit, and spaced apart from the mask pad are further included. The remote control interworked with the communication unit to remotely control the control unit and receives an operating-state signal created from the control unit to display operating-state information of the mask apparatus.

Pursuant to embodiments of the present invention, a remote control connected to the mask pas and spaced apart from the mask pad and a mobile phone interworking with the remote control are further included. The mobile phone generates facial image information through a camera module, checks a skin condition at a specific part on a face by analyzing the facial image information, and transmits the facial skin condition information including skin condition at the specific part on the face to the remote control. The remote control differently provides the heat and far-infrared rays in accordance with the facial skin condition by controlling current supply at the specific part of the mask pad placed on a corresponding face using facial skin condition information.

Pursuant to embodiments of the present invention, the mask pad comprises a far-infrared ray generating pad for generating the heat and far-infrared rays, and a far-infrared ray reflecting pad for reflecting the heat and far-infrared rays. The far-infrared ray reflecting pad includes one of a gold foil sheet, a silver foil sheet, and a complex sheet of gold and silver and makes heat and far-infrared rays evenly spread to a face of a user.

Pursuant to embodiments of the present invention, a fabric facial mask including gold and silver is further included. The fabric facial mask includes a skin condition display unit for displaying a waste condition of the face of the user.

Pursuant to embodiments of the present invention provides a mask apparatus for skin beauty with a complex layer including gold and silver comprising a face mask, a mask pad placed within the face mask and formed by a multi-layered structure of gold and silver, and providing skin beauty effect on a face of a user using heat and far-infrared rays, and a control unit for supplying current and controlling temperature. The mask pad comprises a far-infrared ray generating pad is formed on one side of the face mask and generates heat and far-infrared rays, and a far-infrared ray reflecting pad is formed on one side of the far-infrared ray generating pad and reflects heat and far-infrared rays toward a face of a user.

Pursuant to embodiments of the present invention, the far-infrared ray generating pad comprises a heat generating layer printed for silver paste to have a first pattern, and a far-infrared rays generating layer printed for carbon-based graphite and Carbon Nano Tube (CNT) to have a second pattern on the heat generating layer. The far-infrared ray reflecting pad includes one of a gold foil sheet, a silver foil sheet, and a complex sheet of gold and silver.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

A more complete appreciation of the invention, and many of the attendant advantages thereof, will be readily apparent as the same becomes better understood by reference to the following detailed description when considered in conjunction with the accompanying drawings in which like reference symbols indicate the same or similar components, wherein

FIG. 1 shows an appearance of a mask apparatus for skin beauty according to an embodiment of the present invention;

FIG. 2(a) and FIG. 2(b) are views for explaining a mask apparatus for skin beauty according to another embodiment of the present invention;

FIG. 3 shows a fabric facial mask of the present invention;

FIG. 4 is an exploded view of a face mask according to the present invention;

FIG. 5(a), FIG. 5(b) and FIG. 5(c) are views for explaining a far-infrared ray generating pad of mask pads according to the present invention;

FIG. 6(a) and FIG. 6(b) show a mask pad having a two-layered structure according to an embodiment of the present invention;

FIG. 7 shows a mask pad depending on a face area according to an embodiment of the present invention;

FIG. 8 is a block diagram of a remote control according to an embodiment of the present invention;

FIG. 9 shows ventilation holes formed in a face mask according to an embodiment of the present invention;

FIG. 10 shows a structure of a fabric facial mask according to an embodiment of the present invention;

FIG. 11(a) and FIG. 11(b) show a far-infrared ray reflecting pad covered with a gold foil sheet according to an embodiment of the present invention; and

FIGS. 12 and 13 are photographs for explaining effects depending on a far-infrared ray reflecting pad according to an embodiment of the present invention;

### DETAILED DESCRIPTION

Hereinafter, the present invention will be described in detail with reference to the drawings. In describing the present invention, detailed descriptions related to publicly known functions or configurations will be omitted in order not to obscure the gist of the present invention.

The present invention may, however, be embodied in different forms and should not be construed as limited to the embodiments set forth herein.

Various modifications to the preferred embodiments will be readily apparent to those skilled in the art and the generic principles herein may be applied to other embodiments. Thus, the present invention is not limited to the embodiment shown but is to be accorded the widest scope consistent with the principles and features described herein.

The present invention may, however, be embodied in different forms and should not be construed as limited to the embodiments set forth herein. The same reference numeral is used to refer to like elements throughout.

In the specification, terms such as “include” or “have” should be understood as designating that features, number, steps, operations, elements, parts, or combinations thereof exist and not as precluding the existence of or the possibility of adding one or more other features, numbers, steps, operations, elements, parts, or combinations thereof in advance.

The terminology which is used in common will be used for the purpose of description and not of limitation. Furthermore, terms and words used by the applicant may be used for special cases. In this case, the meaning of terms or words must be understood with due regard to the meaning expressed in the specification rather than taking into account only the basic meaning of the terms and words.

Hereinafter, the technical construction of the present invention will be described in detail with reference to preferred embodiments illustrated in the attached drawings.

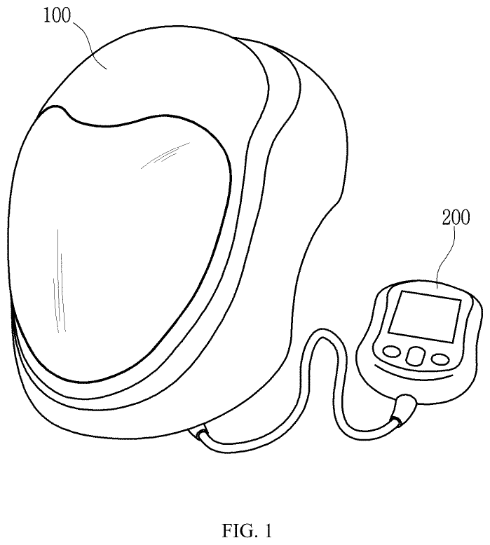

FIG. 1 shows an appearance of a mask apparatus for skin beauty according to an embodiment of the present invention.

The mask apparatus includes a face mask 100 and a remote control 200.

The face mask 100 is capable of providing skin beauty effect such as anti-wrinkling or pore control on the face as well as skin care effect such as blood circulation improvement using heat and far-infrared ray.

For this, the face mask 100 includes a mask pad for generating heat and far-infrared ray therein.

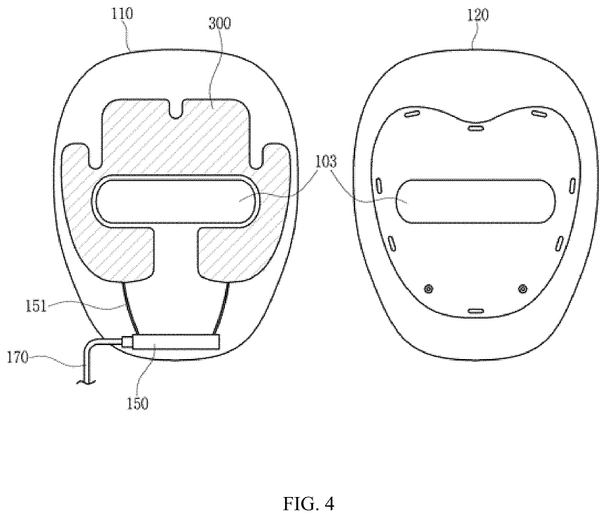

Referring to FIGS. 4 to 7, the mask pad will be described hereinafter.

The remote control 200 performs functions to control a degree of heat and far-infrared ray generation and generation areas of heat and far-infrared ray of the face mask 100.

Users are capable of operating the remote control 200 to control a function of the face mask 100.

In addition, the remote control 200 can automatically control the function of the face mask 100 in order that optimal heat and far-infrared ray to a face skin of an user using face skin condition information of users. In this case, a control unit 250 can be formed in the remote control 200.

The remote control 200 is connected to the face mask 200 by wire. In this case, power supply such as a battery is embedded and power supply is provided to the face mask 100 through a power cable, and thereby reducing electromagnet waves.

Additionally, the remote control 200 can be connected to through a communication an external communication device such as smart phones. The remote control 200 receives face skin condition information of users from the external communication device to control generating heat and far-infrared ray of the face mask 100.

For example, the remote control 200 performs a communication through a near field communication such as Wi-Fi network, Bluetooth, and the like or is connected through a mobile communication such as CDMA/LTE. Smart phones create a face image through a camera module, analyzes the face image to check a face skin condition, and then transfers checked information to the remote control 200. The remote control 200 controls the heat and far-infrared ray functions of the face mask with corresponding to the checked information using the face skin condition information of users from smart phones.

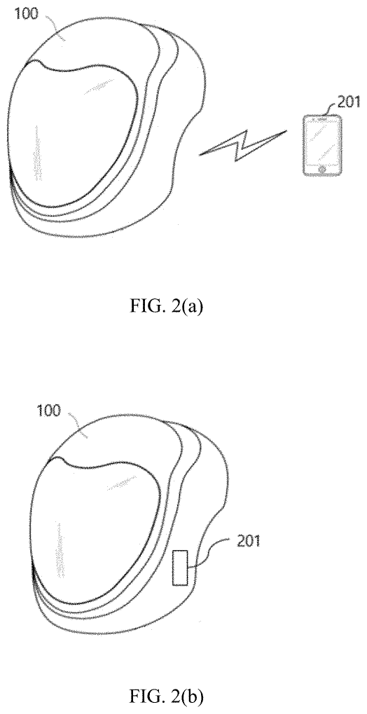

The remote control 200 connected by wire may be omitted. In this case, as shown in FIG. 2(a), a degree of generation and a generation portion of heat and far-infrared rays of the face mask 100 can be controlled through the remote control 210. A battery, a control unit, and a wireless communication unit, which are note shown, may be formed in the face mask 100.

The battery supplies currents to a mask pad. The control unit receives a control signal from the remote control through the wireless communication unit to control current intensity output from the battery and the amount of currents provided to the mask pad.

The wireless communication unit includes a wireless communication device using short-distance wireless communication such as zigbee, bluetooth, and the like, a wireless communication device using WiFi wireless communication, and a wireless communication device using mobile communication such as CDMA/LTE.

The remote control 201 may be a smart phone. If the remote control 201 is a smart phone, it is possible that a software is installed in the smart phone to control the control unit of the face mask.

As shown in FIG. 2(b), instead of the remote control 200 connected by wire, an input unit 202, a battery (not shown), and a control unit (not shown) may be formed. A degree of generation and a generation portion of heat and far-infrared rays can be controlled by operating the input unit 202 formed in the face mask 100. The input unit 202 is an input device for generating a control signal by manual operation such as a button input device, a touch pad input device, a dial input device, and so forth.

Instead of the manual operation, the control unit can be controlled by sounds. For this, a mike (not shown) is included in the present invention. In this case, the control unit can be operated by voice of a user or a predetermined sound.

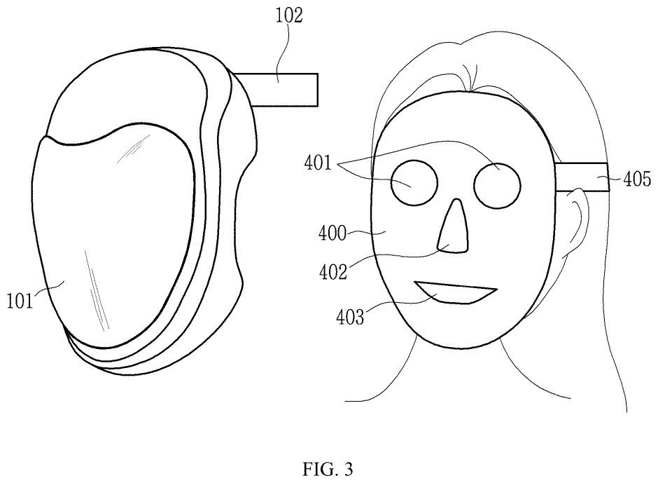

For easy wearing, the face mask 100 is formed to be in contact with a face of an user, and a band 102 (See FIG. 3) is formed on an upper portion of the face mask 100.

In addition, a cover opening periocular for securing eyes 101 is formed on a front part of the face mask 100. The cover 101 has a permeation rate less than or equal to 50% toward outside direction and a reflection rate greater or equal to 70% toward inside.

As shown in FIG. 3, a fabric facial mask 400 containing gold and silver is further included so as to improve skin beauty effect by gold and silver. The fabric facial mask 400 is formed of a fabric containing gold and silver.

The fabric facial mask 400 is wearable and corresponding to a face shape of an user and includes a band 405 (See FIG. 3) wearable by a user. To wear the fabric facial mask 400, various kinds of methods such as velcro, belt, hanging on ears, and the like.

Accordingly, if users put on the fabric facial mask 400, gold and silver contained therein are directly in contact with a face so that blood circulation and skin beauty improvement directly perform a function onto a face skin of users.

By additionally wearing the fabric facial mask 400 on the face mask 100, skin improvement by heat and far-infrared ray as well as blood circulation and skin beauty improvement by gold and silver can be dramatically performed. In other words, removing skin waste and improving blood circulation by gold as well as sterilizing by silver are more effectively applied to a face skin through heat and far-infrared ray.

In this case, as shown in Fig, the fabric facial mask 400 is put on by an user with the band 405 but can be positioned on an inner side of the face mask 100. In this case, a removable configuration with the inner side of the face mask 100 can be added with the fabric facial mask 400. For instance, a Velcro is formed on inner sides of the face mask 100 and the fabric facial mask 400 so that they are removable each other.

In order to secure eyes and breathe, there are holes on portions 401 corresponding to eyes, portions 402 corresponding to a nose, and portions 403 corresponding to a mouth of the fabric facial mask 400.

Referring to FIGS. 4 to 7, a mask pad formed in a face mask will be described in more detail.

The face mask 100 comprises an inner face mask 110 and a face mask cover 120.

The mask pad 300 is adhered to the inner face mask 110 as shown in FIG. 4.

The mask pad 300 is positioned between the inner face mask 110 and the face mask cover 120.

The mask pad 300 is adhered to the inner face mask to emit heat and far-infrared ray to a face of users, thereby providing skin beauty effect.

A view hole 103 is formed to secure eyes in the inner face mask 110 and the face mask cover 120. The mask pad 300 is adhered around the view hole 103 of the inner face mask 110.

A remote control connection part 150 for connecting to the remote control 200 is formed in portions corresponding to a jaw of the inner face mask 110. Currents are supplied through a power line 151 of the remote control connection part 150 to the mask pad 300.

The remote control connection part 150 is electrically connected through a remote control cable 170 to the remote control 200.

If a wireless remote control 201 is employed in the present invention, the remote control connection part 150 includes a wireless communication device and performs communication with the wireless remote control 201 using wireless communication.

The mask pad 300 of the present invention comprises a far-infrared ray generating pad 310 and a far-infrared ray reflecting pad 320. The far-infrared ray generating pad 310 creates heat and far-infrared ray and the far-infrared ray reflecting pad 320 is adhered to the far-infrared ray generating pad 310 to reflect the heat and far-infrared ray from the far-infrared ray generating pad 310 toward a face of users.

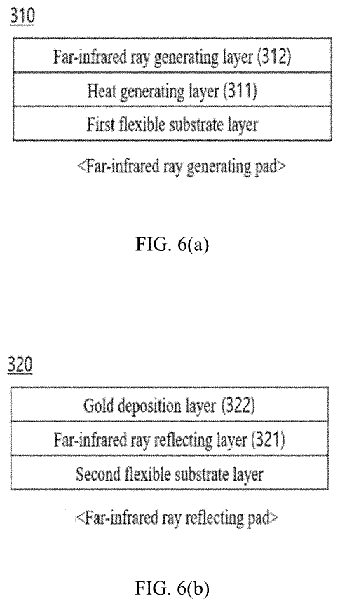

The far-infrared ray generating pad 310 is a planar heater creating heat and far-infrared ray and is formed of a three-layered structure of a first flexible substrate layer, a heat generating layer, and a far-infrared rays generating layer as shown in FIG. 6(a).

The first flexible substrate layer is easily modified according to an outline of the inner face mask 110 (a face outline of users) and formed of flexible materials.

The heat generating layer 311 is formed on the first flexible substrate layer to create heat. The heat generating layer 311 is formed by printing silver paste formed of silver, silver powder, and silver nanowire through a screen printing method to have a first pattern on the first flexible substrate layer. The first pattern is formed using various well known methods as well as a screen printing method. If currents are supplied to the first pattern, heat generates in the first pattern. The first pattern includes silver.

A far-infrared rays generating layer 312 performs a function to convert heat generated from the heat generating layer to far-infrared ray. For this, the far-infrared rays generating layer 312 is formed by printing carbon-based graphite and Carbon Nano Tube (CNT) using a screen printing method to have a second pattern on the heat generating layer 311. The second pattern is formed using various well known methods as well as a screen printing method.

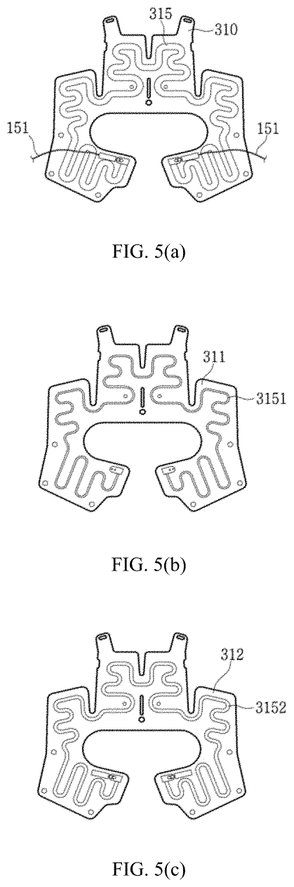

The first pattern 3151 on the heat generating layer 311 and the second pattern 3152 on the far-infrared rays generalayer 312 are overlapped as shown in FIG. 5(b) and FIG. 5(c).

The first and second patterns 3151 and 3152 are overlapped in the same/similar pattern. The overlapped first and second patterns 3151 and 3152 form a heat and far-infrared ray generation part 315 for emitting heat and far-infrared rays.

Referring to FIG. 5(a), FIG. 5(b) and FIG. 5(c), the heat and far-infrared ray generation part 315 emits heat and far-infrared ray of the far-infrared ray generating pad 310. The first pattern 3151 and second pattern 3152 are formed on the heat generating layer 311 and far-infrared rays generating layer 312, which are formed in a two-layered structure, respectively. The first pattern 3151 and second pattern 3152 are overlapped each other to form the heat and far-infrared ray generation part 315.

Heat is generated in the first pattern 3151 of the heat and far-infrared ray generation part 315. Due to the generated heat, the second pattern 3152 is influenced to emit far-infrared ray.

The heat and far-infrared ray generation part 315 is connected to a power line 151 for receiving currents from the remote control 200.

As mentioned above, the first pattern 3151 is formed for silver paste formed of silver, silver powder, and silver nanowire through a screen printing method to have a predetermined pattern and creates heat by receiving currents through the power line 151. The second pattern 3152 is formed for carbon-based graphite and Carbon Nano Tube (CNT) to be overlapped with the first pattern 3151 through a screen printing method to form the second pattern 3152. The second pattern 3152 generates far-infrared rays using heat generated from the first pattern 3151.

That is, when currents are provided to the first pattern 3151 formed of silver paste with high thermal conductivity, heat is created. The created heat is transferred to the second pattern 3152 to create far-infrared rays in the second pattern 3152.

The far-infrared ray reflecting pad 320 reflects heat and far-infrared ray therefrom.

For this, the far-infrared ray reflecting pad 320, as shown in FIG. 6(b), comprises a second flexible substrate layer, a far-infrared ray reflecting layer 321 and a gold deposition layer 322.

The second flexible substrate layer is adhered on the far-infrared ray generating pad 310 and formed of flexible materials.

The far-infrared ray reflecting layer 321 is coated on the second flexible substrate layer by performing a screen printing method or a vacuum deposition method with respect to silver paste formed of silver, silver powder, and silver nanowire, which have high far-infrared ray reflectance. The far-infrared ray reflecting layer 321 has heat and far-infrared ray reflectance property.

The gold deposition layer 322 is formed on the far-infrared ray reflecting layer 321. The gold deposition layer 322 is coated on the far-infrared ray reflecting layer 321 through a deposition method. The gold deposition layer 322 performs a function to effectively progress heat and far-infrared rays toward a face of an user and protect the far-infrared ray reflection layer.

In this case, the gold deposition layer 322 can be omitted.

The far-infrared ray reflecting pad 320 can be formed of one of a silver foil sheet, a gold foil sheet, and a complex sheet of gold and silver.

Therefore, the mask pad 300 is formed with a complex layer including gold and silver to have antibacterial, anti-aging, and improved blood circulation effects.

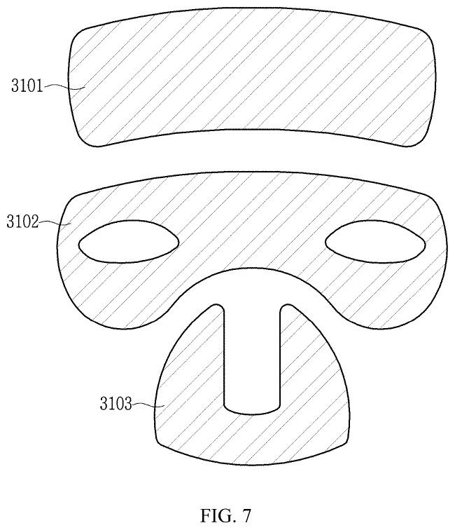

The far-infrared ray generating pad 310 may be constituted in one entity (See FIG. 5(a)) or may be constituted separately for providing different heat and far-infrared ray according to a skin condition on a specific part of a face (See FIG. 7).

The far-infrared ray generating pad 310 comprises a brow-region far-infrared ray generating part 3101, an eye-region far-infrared ray generating part 3102 and a nasolabial-line region far-infrared ray generating part 3103.

The brow-region far-infrared ray generating part 3101 provides heat and far-infrared ray to a brow region to intensively improve wrinkles thereof.

The eye-region far-infrared ray generating part 3102 provides heat and far-infrared ray to an eye region to intensively improve wrinkles around eye and eye circles thereof.

The nasolabial-line region far-infrared ray generating part 3103 provides heat and far-infrared ray to a nasolabial-line region to intensively improve nasolabial folds thereof.

The brow-region far-infrared ray generating part 3101, the eye-region far-infrared ray generating part 3102 and the nasolabial-line region far-infrared ray generating part 3103 receive power supply (currents) from the remote control 200 through an additional power line to provide heat and far-infrared ray to appropriate portions.

When the heat generating layer 311 of the far-infrared ray generating pad 310 is constituted with three components to differently and respectively receive currents, the far-infrared ray generating layer 312 is formed dividedly or in one entity.

If the far-infrared ray generating pad 310 is formed as two or more portions, the far-infrared ray reflecting pad 320 is formed dividedly or in one entity like the far-infrared ray generating pad 310.

As mentioned above, the far-infrared ray generating pad 310 is capable of differently providing heat and far-infrared ray depending on a face skin condition of an user. Accordingly, the mask apparatus of the present invention improves skins more effectively rather than a conventional mask apparatus for providing the same heat and far-infrared ray on a whole face of an user.

In addition, under the same power capacity, needed currents are provided to a required facial area thereby reducing power supply as compared with a conventional face mask apparatus for providing the same currents on a whole face. Battery life can be more extended.

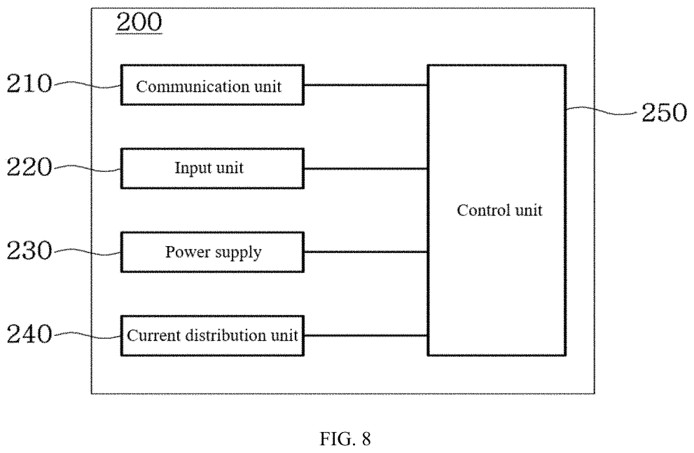

FIG. 8 is a block diagram of a remote control according to an embodiment of the present invention. Referring to FIG. 8, the remote control will be described hereinafter.

The remote controller 200 of the present invention comprises a communication unit 210, an input unit 220, a power supply 230, a current distribution unit 240, and a control unit 250.

The communication unit 210 performs a function to communicate with an external communication device and may be omitted.

The communication unit 210 receives face skin condition information of an user by communicating with a smart phone. For this, the communication unit 210 may include a near field communication such as Wi-Fi network, Bluetooth or a mobile communication such as CDMA/LTE.

The input unit 220 may include a keypad as an input device for controlling the remote controller 200 or a touch panel.

The input unit 220 may include a display device for displaying an input status and an operation status of the remote controller 200.

The power supply 230 supplies power source to the remote controller 230 and the mask pad 300 and may include a battery or a power converting device.

The current distribution unit 240 provides currents to the mask pad 300 and distributes required currents according to a face skin condition of an user to the mask pad 300.

If the far-infrared ray generating pads 310 is formed in two or more portions that are separated, the current distribution unit 240 differently provides currents to corresponding portions.

The control unit 250 controls functions of the remote controller 200 and currents to be provided to the mask pad 300 and the far-infrared ray generating pad 310.

If the control unit 250 receives information about a face skin condition of users, it analyzes and determines the face skin condition. Then, in order to provide optimal skin beauty effect or skin care effect to corresponding face portions, the control unit 250 controls the current distribution unit 240 by calculating a current amount required to provide heat and far-infrared ray to corresponding portions so that a required current is distributed to the corresponding portions.

The remote control 200 may further include a storage unit (not shown) for storing facial skin condition information of an user.

By using the stored facial skin condition information, the control unit 250 calculates the amount of heat and required onto a face to be capable of controlling the current distribution unit 240 in order that corresponding heat and far-infrared ray are provided to corresponding portions.

Pursuant to an embodiment of the present invention, heat and far-infrared ray are provided to a face of a user by communicating with an external communication device assuming that the external communication device is a smart phone.

The smart phone generates facial image information through a camera module, check a skin condition at each of parts on a face by analyzing the facial image information, and transmits facial skin condition information including skin condition at the specific part on the face to the remote control 200. For performing these operations, the smart phone may have a program interworking with the remote controller 200 of the present invention.

In accordance with another embodiment of the present invention, the smart phone transmits the generated facial image information to the remote control 200, and the control unit 250 of the remote control 200 analyzes the facial image information to be capable of checking a facial skin condition of an user.

The control unit 250 of the remote control 200 controls the current distribution unit 240 to provide currents suitable to corresponding face portions in order that optimal heat and far-infrared ray are supplied for skin beauty or skin care on the corresponding face portions using facial skin condition information received from the smart phone or facial skin condition information generated from the control unit 250.

In other words, the control unit 250 is capable of differently providing currents to the brow-region far-infrared ray generating part 3101, the eye-region far-infrared ray generating part 3102 and the nasolabial-line region far-infrared ray generating part 3103 in order that heat and far-infrared ray suitable to each of corresponding portions are provided.

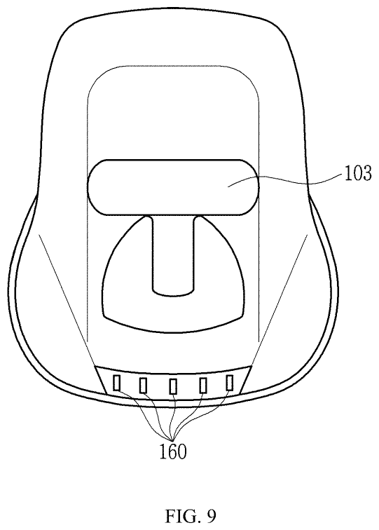

FIG. 9 shows ventilation holes formed in a face mask according to an embodiment of the present invention.

In the mask apparatus for skin beauty with the complex layer including gold and silver, holes 160 are formed in portions corresponding to a jaw of the inner face mask 110. Through these holes, users can freely breathe and talk while wearing the face mask 100.

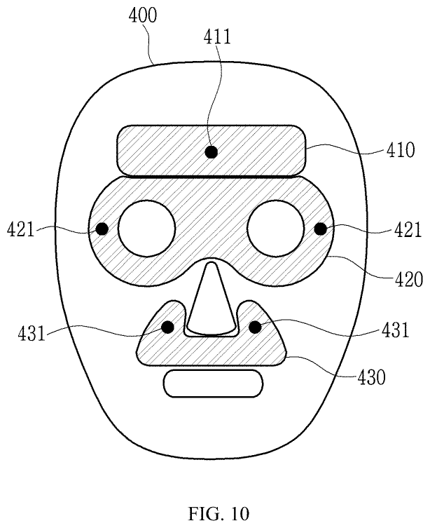

FIG. 10 shows a structure of a fabric facial mask according to an embodiment of the present invention. Referring to FIG. 10, the fabric facial mask 400 of the present invention, as mentioned above, gold and silver are additionally contained in fabric materials and a skin condition displaying units 411, 421, and 431 are further included.

Gold and silver are distributed to a fabric with uniform density. As shown in FIG. 10, gold and silver with high density can be distributed on face skin portions required to be more improved, rather than other face skin portions. For instance, gold and silver with high density are distributed on the brow region 410, the eye region 420, and the nasolabial-line region 430 rather than other portions.

Also, if the far-infrared ray generating pad 310 is formed of a plurality of the brow-region far-infrared ray generating part 3101, the eye-region far-infrared ray generating part 3102, and the nasolabial-line region far-infrared ray generating part 3103, the density of gold and silver is higher on the corresponding portions of the fabric facial mask 400 than other portions.

The fabric facial mask 400 contains silver to directly sterilize microorganism onto a face of an user. A gold pad, a silver pad and a gold/silver pad are removable on a whole fabric facial mask. Furthermore, the gold pad, the silver pad, and gold/silver pad of the face mask 400 are affixed to a wanted face portion of users to dramatically enhance blood circulation and skin beauty effect by gold and silver.

The skin condition displaying units 411, 421, and 431 is capable of displaying a skin waste condition.

The skin condition displaying units 411, 421, and 431 are formed of materials whose colors are changed if they are combined with poisonous component in a body of human being. As a result, it is possible to find a skin waste condition by checking a changed color.

If skin condition displaying units 411, 421, and 431 are formed of litmus papers, their colors are changed from original instruction color to another instruction color depending on skin waste conditions of an user such as acidic or alkali so that it is possible to check the skin waste condition and a degree thereof. That is, the replacement cycle of the fabric facial mask 400 can be notified through the skin condition displaying units 411, 421, and 431.

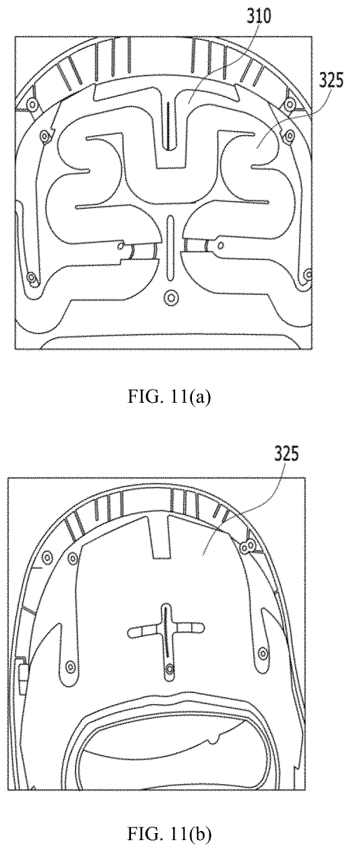

FIG. 11(a) and FIG. 11(b) show a far-infrared ray reflecting pad covered with a gold foil sheet according to an embodiment of the present invention. Referring to FIG. 11(a), the far-infrared ray reflecting pad 320 is formed of one reflecting sheet 325 and adhered on the far-infrared ray generating pad 310.

The reflecting sheet 325 is one of a gold foil sheet, a silver foil sheet, and a complex sheet of gold and silver, which perform a function to reflect heat and far-infrared rays toward a face of an user.

The reflecting sheet 325 is formed on a partial area (See FIG. 11(a)) a whole area (See FIG. 11(b)) of the far-infrared ray generating pad 310.

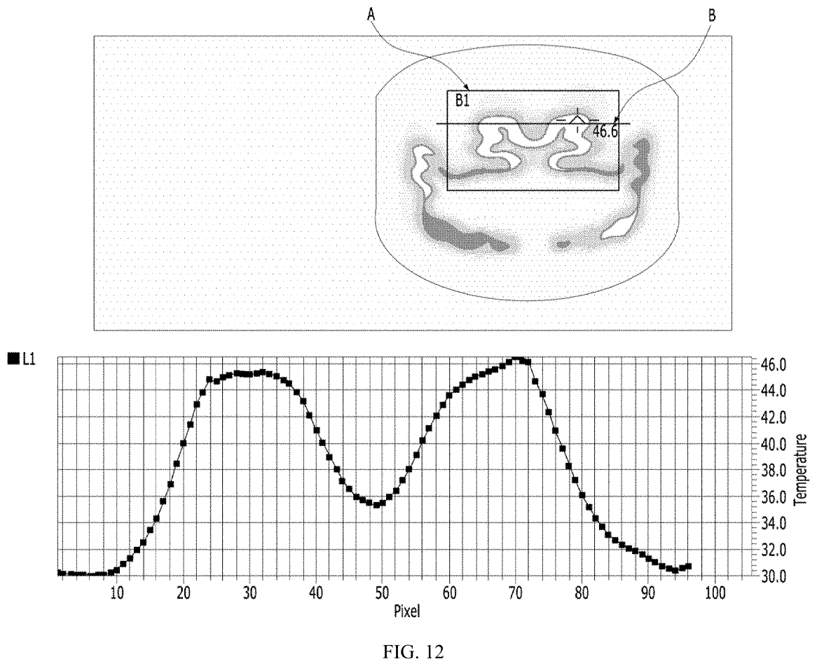

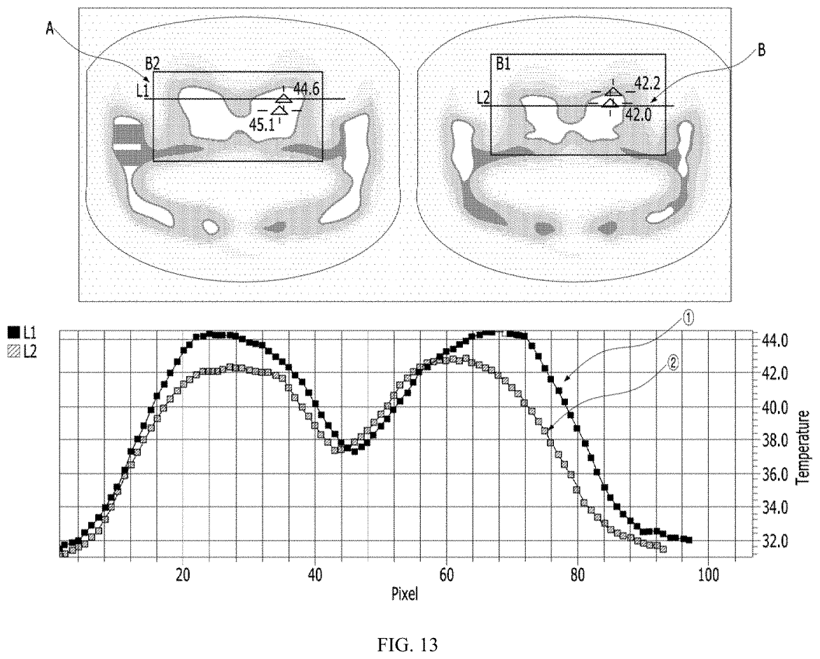

Referring to FIGS. 12 and 13, the effects according to the far-infrared ray generating pad 310 will be described.

As mentioned above, the far-infrared ray generating pad 310 performs a function to reflect heat and far-infrared rays toward a face of an user.

An experiment was performed with respect to a product with a far-infrared ray generating pad and a product without a far-infrared ray generating pad. FIG. 12 shows a result using a gold foil sheet, and FIG. 13 shows a result using a gold foil sheet. In FIG. 13, a gold sheet (See FIG. 11(b)) having a thickness of 20 μm or 35 μm as a far-infrared ray reflecting pad. To compare a temperature at the same point, a temperature of a brow region of an user was compared.

Referring to FIGS. 12 and 13, the maximum and minimum temperatures were measured on a straight line (B) within the brow region (A) marked by rectangular line.

Referring to FIG. 12, the maximum temperature in the brow region was approximately 46.6° C., and the temperature deviation between the maximum and minimum temperatures on the straight line (B) was approximately 11.2.

Referring to FIG. 13, if the thickness of the gold foil sheet was 20 μm, the maximum temperature in the brow region was approximately 46.6° C., and the temperature deviation between the maximum and minimum temperatures on the straight line (B) was approximately 7.2° C. If the thickness of the gold foil sheet was 35 μm, the maximum temperature in the brow region was approximately 42.8° C., and the temperature deviation between the maximum and minimum temperatures on the straight line (B) was approximately 5.7.

The experimental results of FIGS. 12 and 13 were shown in the following Table 1.

TABLE

| | | Product with gold | |
|---|---|---|---|
| | Product | foil sheet (FIG. 13) | |
| | without gold | \textcircled{1} Thickness | \textcircled{2} Thickness |
| | foil sheet (FIG. 12) | 20 um | 35 um |
| Max. Temp. | 46.6 | 44.6 | 42.8 |
| Temp. Deviation | 11.2 | 7.2 | 5.7 |

As shown in Table 1, it is found that heat and far-infrared rays were uniformly distributed on a face of an user by employing the far-infrared ray reflecting like the present invention.

The features of the mask apparatus for skin beauty using the composite including gold and silver will be described hereinafter.

Gold is harmless to human body and highly conductive to electricity. In specifically, pure gold normalizes ion mobility to activate blood circulation, skin biological activity, and cell regeneration. In other words, pure gold helps to promote blood circulation as well as to rapidly remove wastes by vein and lymphatic system. In addition, when blood goes by lung, hemoglobin is linked with oxygen to capillaries of dermis layer. In this case, gold helps to rapidly generate skins by facilitating a circulation cycle of skins.

The positive charge of gold accelerates hormone secretion to maintain skin tightening and relax mind and body. Various functions of gold such as blood circulation promotion, generation promotion, purification, and mid and body relaxation undefinedly described in ancient texts are related with bioelectric currents.

In addition, silver being natural perfect antibiotic does not accompany any kind of side effects and kills virus. That is, silver emits an anion to enhance resistance with respect to wastes and disease. Also, silver improves metabolic to smooth physiological function of human body and is effective in growth and cell generation of human body by removing atmosphere environment pollutions.

Therefore, by directly contacting gold and silver on a skin of users and applying heat and far-infrared ray to gold, problem-relating skins such as sensitive skins, aged bad skins, dryness skins, unclear color skins, and skins with infections are generated into toned and healthy skins through skin biological activity such as blood circulation and waste removal and cell generation function by improving the activity of gold and silver.

According to an embodiment of the present invention, the mask apparatus for skin beauty is capable of providing skin beauty effect such as anti-wrinkling or pore control on the face as well as skin care effect such as blood circulation improvement using heat and far-infrared ray.

In addition, the mask apparatus for skin beauty includes a complex layer including gold and silver to have antibacterial and anti-oxidation effects. Since the mask apparatus for skin beauty uses heat and far-infrared ray, it is capable of improving skin blood circulation and inhibiting age of skin.

Furthermore, a mask pad or a facial fabric mask including gold and silver are worn on a face of an user to additionally uses heat and far-infrared ray. As a result, antibacterial and sterilization effects by silver with heat and far-infrared ray are better than by only silver without heat and far-infrared ray. Skin anti-oxidation, skin activation, and skin blood circulation improvement by gold with heat and far-infrared ray are also better than by only gold without heat and far-infrared ray.

In addition, the mask apparatus for skin beauty is capable of providing different heat and far-infrared ray according to a face part of users so that the heat and far-infrared ray are effectively and differently provided to a corresponding part depending on a facial skin condition of the users. Accordingly, the mask apparatus of the present invention more effectively improves skins than a conventional mask apparatus for providing the same heat and far-infrared ray on a whole face of users.

In addition, according to the mask apparatus for skin beauty of the present invention, needed currents are provided to required facial area to have enhanced energy efficiency as compared with a conventional face mask apparatus for providing the same currents on a whole face.

All such changes, modifications, variations and other uses and applications which do not depart from the spirit and scope of the invention are deemed to be covered by the invention which is limited only by the claims which follow.

What is claimed is:

1. A mask apparatus for skin beauty comprising:
a face mask;
a mask pad placed within the face mask and providing skin beauty effect on a face of a user using heat and far-infrared rays; and
a control unit for capable of differently providing a current to a specific part of the mask pad and differently emitting the heat and far-infrared rays onto the face of the user.

2. The mask apparatus of claim 1, further comprising a remote control connected to the mask pad and spaced apart from the mask pad, wherein the control unit is formed in the remote control and currents are provided through the remote control to the mask pad.

3. The mask apparatus of claim 2, further comprising a power supply formed in the remote control.

4. The mask apparatus of claim 1, further comprising a battery formed in the face mask, wherein the control unit is formed in the face mask and differently provides currents from the battery to the specific part of the mask pad to differently emit the heat and far-infrared rays onto the face of the user.

5. The mask apparatus of claim 1, further comprising a fabric facial mask including gold and silver,
wherein the fabric facial mask removes skin wastes and improves a blood circulation through the heat and far-infrared rays from the mask pad by gold and directly performs a sterilizing function by silver on a facial skin.

6. The mask apparatus of claim 1, wherein the mask pad comprises:
a far-infrared ray generating pad for generating the heat and far-infrared rays; and
a far-infrared ray reflecting pad for reflecting the heat and far-infrared rays,
wherein the far-infrared ray generating pad is formed on one side of the face mask and the far-infrared ray reflecting pad is formed on one side of the far-infrared ray generating pad, and
wherein the far-infrared ray reflecting pad reflects the heat and far-infrared rays created from the far-infrared ray generating pad toward the face of the user.

7. The mask apparatus of claim 6, wherein the far-infrared ray generating pad is a planar heater and comprises:
a first flexible substrate layer;
a heat generating layer printed on the first flexible substrate layer for silver paste to have a first pattern and generating heat when currents are provided; and
a far-infrared rays generating layer printed for carbon-based graphite and Carbon Nano Tube (CNT) to have a second pattern on the heat generating layer and generating far-infrared rays using heat from the heat generating layer.

8. The mask apparatus of claim 6, wherein the far-infrared ray generating pad includes two or more far-infrared ray generating parts, and
wherein the two or more far-infrared ray generating parts are separated each other.

9. The mask apparatus of claim 6, wherein the far-infrared ray reflecting pad includes a second flexible substrate layer and a far-infrared ray reflecting layer in which silver paste is formed on the second flexible layer.

10. The mask apparatus of claim 9, further comprising a gold deposition layer formed on the far-infrared ray reflecting layer.

11. The mask apparatus of claim 1, further comprising an input unit for inputting a control signal of the control unit, wherein the input unit generates the control signal on a manual operation or sound.

12. The mask apparatus of claim 1, further comprising a communication unit and a remote control connected to the mask pad, including a control unit, and spaced apart from the mask pad, and
wherein the remote control interworked with the communication unit to remotely control the control unit and receives an operating-state signal created from the control unit to display operating-state information of the mask apparatus.

13. The mask apparatus of claim 1, further comprising a remote control connected to the mask pas and spaced apart from the mask pad and a mobile phone interworking with the remote control, and
wherein the mobile phone generates facial image information through a camera module, checks a skin condition at a specific part on a face by analyzing the facial image information, and transmits the facial skin condition information including skin condition at the specific part on the face to the remote control, and
wherein the remote control differently provides the heat and far-infrared rays in accordance with the facial skin condition by controlling current supply at the specific part of the mask pad placed on a corresponding face using facial skin condition information.

14. The mask apparatus of claim 1, wherein the mask pad comprises:
a far-infrared ray generating pad for generating the heat and far-infrared rays; and
a far-infrared ray reflecting pad for reflecting the heat and far-infrared rays,
wherein the far-infrared ray reflecting pad includes one of a gold foil sheet, a silver foil sheet, and a complex sheet of gold and silver and makes heat and far-infrared rays evenly spread to a face of a user.

15. The mask apparatus of claim 1, further comprising a fabric facial mask including gold and silver, wherein the fabric facial mask includes a skin condition display unit for displaying a waste condition of the face of the user.

16. A mask apparatus for skin beauty with a complex layer including gold and silver comprising:
a face mask;
a mask pad placed within the face mask, formed by a multi-layered structure of gold and silver, and providing skin beauty effect on a face of a user using heat and far-infrared rays; and
a control unit for supplying current and controlling temperature,
wherein the mask pad comprises a far-infrared ray generating pad is formed on one side of the face mask and generates heat and far-infrared rays; and
a far-infrared ray reflecting pad is formed on one side of the far-infrared ray generating pad and reflects heat and far-infrared rays toward a face of a user.

17. The mask apparatus of claim 16, wherein the far-infrared ray generating pad comprises:
a heat generating layer printed for silver paste to have a first pattern; and
a far-infrared rays generating layer printed for carbon-based graphite and Carbon Nano Tube (CNT) to have a second pattern on the heat generating layer,
wherein the far-infrared ray reflecting pad includes one of a gold foil sheet, a silver foil sheet, and a complex sheet of gold and silver.
